(19) United States
(12) Patent Application Publication
(10) Pub. No.: US 2019/0125790 A1
(43) Pub. Date: May 2, 2019

(54) USE OF HYPOCHLOROUS ACID AS A TOPICAL ANTIMICROBIAL

(71) Applicant: BIIOSMART TECHNOLOGIES LLC, Miami, FL (US)

(72) Inventor: Danyel Armando ARENAS, Bucaramanga (CO)

(21) Appl. No.: 16/096,919

(22) PCT Filed: Apr. 27, 2017
(86) PCT No.: PCT/US2017/029748

§ 371 Date: Oct. 26, 2018

Related U.S. Application Data
(60) Provisional application No. 62/328,553, filed on Apr. 27, 2016.

(51) Int. Cl. A61K 33/20; A61K 9/70; A61K 9/00; A61P 31/22; A61P 31/18; A61P 31/20; A61P 31/14; A61P 31/04; A61K 9/08

(52) U.S. Cl. CPC A61K 33/20; A61K 9/70; A61K 9/0014; A61P 31/22; A61K 9/08; A61P 31/20; A61P 31/14; A61P 31/04; A61K 9/0034; A61P 31/18

(57) ABSTRACT

New methods for the promotion of general sexual hygiene in a subject is provided by taking advantage of the safety and antimicrobial strength of hypochlorous acid in the form of a composition used as a sexual skin cleanser. These methods include the elimination of contact-to-contact STDs in a subject before and after sexual contact, and cleansing of pre- and post-sexual activity transmitted microorganisms.

### BACKGROUND

Human sexual health is extremely important in a changing world. Every day, sexually transmitted diseases (STDs) spread without borders and compromise health and life of thousands of people. The World Health Organization (WHO) reports that more than thirty different bacteria and viruses are known to be transmitted through sexual contact. Barrier methods such as condoms have been extensively promoted since 1960s, however dirty or contaminated skin before and after sexual intercourse can carry on its surface viruses and bacteria that can lead to contamination and infection.

The use of topical antiseptic solutions (e.g. povidone iodine, chlorhexidine, hydrogen peroxide) has been recommended for wound care and compromised skin for years. However, due to low biocompatibility with sensitive skin, toxicity in repeated uses, and adverse effects, daily use of these solutions for genital skin cleansing and to cleanse the genital area during pre- and post-sexual activity is not recommended.

Thus, there remains a need for new topical antimicrobial formulations and cleansing methods that are effective and safe for repeated uses to cleanse the skin exposed to sexual contact, to promote general sexual hygiene, and to prevent skin-to-skin STDs.

### SUMMARY

In an embodiment is a method for promoting sexual hygiene in a subject, comprising: topically applying an effective amount of a hypochlorous acid composition to the genitals and surrounding skin of a subject exposed to sexual contact, wherein the topically applying occurs before and immediately after sexual activity; and allowing the hypochlorous acid composition to remain on the skin.

In another embodiment, is a method for eliminating contact-to-contact STDs in a subject, comprising topically applying an effective amount of a hypochlorous acid composition to the genitals and surrounding skin of a subject exposed to sexual contact/fluids, wherein the topically applying occurs before and immediately after sexual activity; and allowing the hypochlorous acid composition to remain on the skin for a time sufficient to kill contact-to-contact STDs and/or to inhibit absorption of a contact-to-contact STD into the subject via the skin.

In yet another embodiment is a method of cleansing pre- and post-sexual activity transmitted microorganisms, pathogens, viruses, bacteria, and/or bacterial spores, comprising: topically applying an effective amount of a hypochlorous acid composition to the genitals and surrounding skin of a subject exposed to sexual contact, wherein the topically applying occurs before and immediately after sexual activity; and allowing the hypochlorous acid composition to remain on the skin for a time sufficient for a greater than 99% reduction in microorganisms, pathogens, viruses, bacteria, and/or bacterial spores before and after sexual contact.

These and other features and characteristics are more particularly described below.

### DETAILED DESCRIPTION

The use of hypochlorous acid (HOCl) for antimicrobial capabilities is well established in the scientific literature and has been used as a wound healing solution for years. Herein are described new methods for the promotion of general sexual hygiene in a subject by taking advantage of the safety and antimicrobial strength of hypochlorous acid in the form of a composition used as a sexual skin cleanser. These methods include the elimination of contact-to-contact STDs in a subject before and after sexual contact, and cleansing of pre- and post-sexual activity transmitted microorganisms.

Hypochlorous acid is the major inorganic bactericidal compound in innate immunity. Hypochlorous acid is an endogenous molecule which is produced by neutrophils and macrophages as part of the innate immune cascade response to an invading pathogen into a process called “Respiratory Burst”. The compound acts as a powerful oxidant and potent microbicidal agent within cellular immune response, with a high barrier to resistance. As an antimicrobial hypochlorous acid induces, through oxidation and chlorination, damage in important microbial cell wall components such as peptidoglycan. It can also cause oxidation and chlorination in mitochondria and ATP transporters. [Source: Wang et al. J. Burns Wounds, 2007 6: 65-79.] Hypochlorous acid may also neutralize harmful endo and exo toxins as lipopolysaccharides (LPS) and gingipains. HOCl, through oxidation processes, stimulate the production of cellular growth factors, such as insulin-like growth factor, epidermal growth factor, keratinocyte growth factor (also called FGF-7), FGF-1, FGF-2, TGF-β, PDGF, vascular endothelium growth factor, connective tissue growth factor as well many growth factors associated to oral health. Furthermore, hypochlorous acid can activate the transforming growth factor TGF-β, a reparative mediator that promotes tissue repair and fibrosis.

Hypochlorous acid can also cause oxidation and chlorination of the amino acid taurine to yield taurine N-chloramine (TauCl). Oxidation reactions on taurine are more rapid than chlorination reactions and involve thioether and/or thiol groups of proteins. The formation of TauCl seems to play an important role in the regulation of the proteolytic enzymes and metalloproteinases, reducing the impact of these enzymes on the tissue destruction.

Contact-to-contact STDs are passed through direct skin-to-skin contact, where no exchange of bodily fluids is necessary for transmission of the disease. Absorption of skin-to-skin can take up to sixty minutes. Condoms do not fully protect from contact-to-contact STDs.

Use of a hypochlorous acid composition sexual cleanser product by topical application allows for the prevention of contamination with most common bacteria and viruses associated with skin-to-skin contact sexually transmitted diseases in pre- and post-sexual activity settings such as viruses, bacteria, bacterial spores, and fungi, thereby promoting general sexual hygiene. Use of the cleanser product inhibits absorption of STDs into body via the skin and kills STDs at 99.999% efficacy. Thus, topical application of a hypochlorous acid composition significantly reduces or prevents transmission of skin-to-skin STDs.

Exemplary contact-to-contact STDs include herpes type 2 (HSV-2, or genital herpes), human papillomavirus (HPV), syphilis, trichomoniasis, molluscum contagiosum, Hepatitis B and C virus, HIV, and the like. Use of the hypochlorous acid composition can further minimize or prevent the contamination of other microorganisms, pathogens, viruses, bacteria and/or bacterial spores including Aspergillus niger, Bovine Diarrhea virus, Campylobacter rectus, Candida albicans, Enterobacter cloacae, Enterococcus faecalis, Enterococcus faecium, Escherichia coli, Hepatitis B and C virus, Herpes simplex virus type-2 and type-1, human immunodeficiency virus (HIV), human papilloma virus (HPV), Klebsiella oxytoca, Klebsiella pneumoniae, methicillin-resistant staphylococcus aureus, trichomoniasis, molluscum contagiosum, Pseudomona aeruginosa, Salmonella enteritidis, Staphylococcus aureus, Staphylococcus epidermidis, Streptococcus pyogenes, syphilis, Treponema pallidum, Trichophyton mentagrophytes, vancomycin-resistant enterococci, or a combination thereof.

In an embodiment, a method for minimizing or preventing contact-to-contact STDs and infections, including Herpes and HPV, comprises topically applying an effective amount of a hypochlorous acid composition to the genitals and surrounding skin of a subject exposed to sexual contact, wherein the topically applying occurs before and immediately after sexual activity; and allowing the hypochlorous acid composition to remain on the skin for a time effective to minimize or prevent infection in the subject. As used herein “immediately after sexual activity” means within about 10 minutes, more specifically within 5 minutes of sexual intercourse or other sexual activity that exposes the subject to a contact-to-contact STD.

An effective amount of the hypochlorous acid composition can be determined by one having ordinary skill in the art without undue experimentation and takes into consideration various factors such as surface area of the skin exposed to the contact-to-contact STDs or other unwanted microorganisms, pathogens, viruses, bacteria, and/or bacterial spores; amount of contaminated foreign material present on the skin; and the like. In an embodiment, the hypochlorous acid composition comprising about 0.005 to about 0.06% hypochlorous acid by volume, specifically about 0.01 to about 0.05% hypochlorous acid by volume, and more specifically about 0.02 to about 0.046% hypochlorous acid by volume, and yet more specifically about 0.03 to about 0.04% hypochlorous acid by volume based on the total volume in an appropriate liquid vehicle.

In a general embodiment, the hypochlorous acid composition can be formulated as a sexual cleanser product used for the topical cleansing of the genital skin area to remove foreign material contaminated with bacteria and viruses associated with skin-to-skin contact sexually transmitted diseases during sexual intercourse (pre and post activity) to promote general sexual hygiene. Use of the hypochlorous acid composition for cleansing and removal of microorganisms and foreign material involves topical application of the composition to skin, but no rinsing is required.

In an embodiment, the hypochlorous acid composition comprises hypochlorous acid and water. In another embodiment, the hypochlorous acid composition consists essentially of hypochlorous acid and water. In yet another embodiment, the hypochlorous acid composition consists of hypochlorous acid and water. In yet another embodiment, the hypochlorous acid composition comprises hypochlorous acid and water and is substantially free of one or more of sodium hypochlorite (NaHClO₃), sodium chloride (NaCl), chlorine (Cl₂), ozone (O₃), hydrogen peroxide (H₂O₂), sodium hydroxide (NaOH), or a combination thereof.

Hypochlorous acid can be prepared using techniques and procedures known in the art, including chemical and electrochemical techniques. Exemplary processes include
Hydrolysis of chlorine gas Cl₂+H₂O→HOCl+H⁺+Cl⁻; or
Electrolysis of a salt solution 2Cl⁻+2e⁻→Cl₂+H₂O→HOCl+H⁺+Cl⁻; or
Acidification of hypochlorite OCl⁻+H⁺→HOCl.

The hypochlorous acid composition can be formulated for administration topically to a subject, e.g., by the direct application of the product to the skin of the subject, particularly the male and female genital area and surrounding skin. The composition may be formulated as a liquid, gel, lotion, cream, a foam, or liquid spray. Such formulations may be produced in a conventional manner using appropriate carriers which are well known to a person skilled in the art. Alternatively, the hypochlorous acid composition can be used to prepare a product in the form of a pre-moistened towel or wipe saturated with the composition using processes and materials known in the art.

The hypochlorous acid composition can be in the form of an aqueous solution comprising water. The water may be deionized, filtered to remove impurities including metals and organic carbon, purified by reverse osmosis, purified by distillation, or a combination thereof. Purified water may be prepared by a process selected from the group consisting of sodium cation exchange, hydrogen cation exchange, reverse osmosis, activated carbon treatment, UV light treatment, UV-C light treatment, ozone treatment, chlorination, ultrafiltration, nanofiltration, electrodialysis, or a combination thereof.

The hypochlorous acid composition may have a pH from about 2 to about 6, specifically about 3 to about 5, and yet more specifically about 3.5 to about 4.5. In an embodiment, the product is pH balanced for skin compatibility. In an embodiment, the product has a pH of about 5.2 to about 6.0. A hypochlorous acid composition having a pH between 3.5 and 4.5 is highly effective in the chlorination of microorganisms, with an effectiveness occurring within 30 seconds.

The hypochlorous acid composition has an oxidation-reduction potential that ensures its antimicrobial effectiveness. The oxidation-reduction potential of the hypochlorous acid composition can be greater than 800 millivolts (mV), specifically about 900 to about 1300 mV, more specifically about 1000 to about 1250 mV, and still more specifically about 1100 to about 1200 mV allowing it to effectively oxidize the plasma membrane of microorganisms leading to microorganism death.

The hypochlorous acid composition can be prepared into a formulation further containing one or more additional agents that are stable to hypochlorous acid and are pharmaceutically acceptable. Such additional agents may include one or more of a buffer, a colorant, a defoamer, a fragrance, a gel forming agent, a pH adjusting agent, a preservative, an additional solvent, a stabilizer, a surfactant, a thickener, and the like.

The hypochlorous acid composition may be adjusted for pH using a pH adjusting agent. Exemplary pH adjusting agents include carbon dioxide, alkali metal carbonate, alkali metal bicarbonate, alkali metal silicates, alkali metal hydroxide, alkali phosphate salt, alkaline earth phosphate salt, alkali borate salt, hydrochloric acid, nitric acid, sulfuric acid, alkali metal hydrogen sulfate, acetic acid, vinegar from various sources, other carboxylic acids, polycarboxylates, organic sulfonic acids, sulfamic acid, amine, alkyl amine, dialkyl amine, trialkyl amine and the like.

Gelling agents include silicone oil such as dimethicone (polydimethylsiloxane), hydrogel forming agents such as a natural or synthetic clay, a cellulosic, a carbomer, a combination thereof, and the like.

Disclosed herein is a hypochlorous acid sexual cleanser product comprising a hypochlorous acid composition comprising about 0.005 to about 0.06% hypochlorous acid by volume, specifically about 0.01 to about 0.05% hypochlorous acid by volume, more specifically about 0.02 to about 0.046% hypochlorous acid by volume, and yet more specifically about 0.03 to about 0.04% hypochlorous acid by volume based on the total volume in an appropriate liquid vehicle. In an embodiment, the liquid vehicle is water. In an embodiment, the hypochlorous acid composition comprises a concentration of hypochlorous acid, e.g. about 0.05% by volume or about 0.046% by volume, that allows it to act as an antimicrobial immediately and effectively thus preventing contamination or bacterial growth.

In an embodiment, the hypochlorous acid composition is an aqueous composition formulated topical spray applied before and after sexual activity. The hypochlorous acid sexual cleanser product is non-irritating, non-sensitizing, non-toxic, and biocompatible to the skin and tissue. For example, application of about 0.046 to about 0.05% hypochlorous acid solution composition as a topical sexual cleansing product with a specific focus on male and female genital area was found to be safe and effective.

The hypochlorous acid composition can be in the form of a topical solution skin cleanser product for the cleansing of skin, specifically designed to remove foreign material non contaminated or contaminated with most common bacteria and viruses present on male and female genital skin environment in order to clean and prevent skin-to-skin contamination and to promote general sexual hygiene.

In an embodiment, the hypochlorous acid composition is prepared as a ready-to-use liquid for easy application on skin. The liquid can be used in a spray device, such as a pump spray device, for convenient application to saturate the surface of the skin.

The hypochlorous acid composition can be contained and packaged in any type of container material that is stable to hypochlorous acid, for example, glass, acrylonitrile-butadiene-styrene (ABS), polycarbonate, high density polyethylene, low density polyethylene, high density polypropylene, low density polypropylene, polyethylene terephthalate, polyvinylchloride, and the like.

The hypochlorous acid composition exhibits antimicrobial activity for a variety of microorganisms found in the genital area including bacteria, e.g. Pseudomonas aeruginosa, Escherichia coli, Staphylococcus aureus, etc.; and fungi, e.g., Candida albicans, Aspergillus niger, etc. The product prevents contamination within the genital topical/skin environment.

In one embodiment, the hypochlorous acid composition is prepared into a sexual cleanser product containing a concentration of about 0.01 to 0.05%, specifically about 0.02 to about 0.046% hypochlorous acid by volume in electrolyzed water with a mild, base adjusted pH of 5.0 (+/−0.2) for compatibility with the normal pH of the skin. Further within this embodiment, the hypochlorous acid sexual cleanser product is non-toxic, non-irritating, and biocompatible; and further non-staining, non-oily, non-foaming, steroid-free, and/or antibiotic-free.

As used herein, the term “skin” includes the epidermis and dermis.

In an embodiment, the hypochlorous acid composition is prepared into a sexual cleanser product containing a concentration of about 0.04 to about 0.046% hypochlorous acid by volume in electrolyzed water with a pH of about 5.0 to about 6.0, specifically a pH of about 5.2 to about 6.0.

The following example are merely illustrative of the methods disclosed herein and are not intended to limit the scope hereof.

### EXAMPLES

### Example 1 Antimicrobial Data

Table 1. below presents hypochlorous acid efficacy against some of the most common contaminants and pathogens found in skin and genital area. A 0.01% aqueous hypochlorous acid solution had a 99.99% efficacy on contact at 30 seconds when used as a skin cleanser for the genital area in pre- and post-sexual activity.

TABLE 1

| | HOCl | Log | | % |
|---|---|---|---|---|
| Microorganism | concentration | Reduction | | Reduction |
| Staphylococcus aureus | 0.01% | >6.1 | Log₁₀ | 99.999% |
| Staphylococcus epidermidis | 0.01% | >5.8 | Log₁₀ | 99.999% |
| Enterococcus faecalis | 0.01% | >6.5 | Log₁₀ | 99.999% |
| Salmonella enteritidis | 0.01% | >6.4 | Log₁₀ | 99.999% |
| Escherichia coli | 0.01% | >6.59 | Log₁₀ | 99.99% |
| Pseudomona aeruginosa | 0.01% | >5.94 | Log₁₀ | 99.99% |
| S. pyogenes | 0.01% | >6.12 | Log₁₀ | 99.99% |
| Klebsiella oxytoca | 0.01% | >5.50 | Log₁₀ | 99.99% |
| Enterobacter cloacae | 0.01% | >5.76 | Log₁₀ | 99.99% |
| Campylobacter rectus | 0.01% | >5.96 | Log₁₀ | 99.99% |
| Candida albicans | 0.01% | >5.98 | Log₁₀ | 99.99% |
| Enterococcus. faecium | 0.01% | >6.21 | Log₁₀ | 99.99% |
| Human papilloma virus | 0.01% | >6.0 | Log₁₀ | 99.99% |
| HIV (non-glp) | 0.01% | >6.1 | Log₁₀ | 99.999% |
| Herpes simplex virus | 0.01% | >5.8 | Log₁₀ | 99.99% |

Table 2. below presents hypochlorous acid efficacy against additional common contaminants and pathogens found in skin and genital area. A 0.01 to 0.05% aqueous hypochlorous acid solution has a 99.99% efficacy on contact at 30 seconds when used as a skin cleanser for the genital area in pre- and post-sexual activity.

TABLE 2

| | Efficacy at 30 seconds | | | |
|---|---|---|---|---|
| | | | Control of Innoculum | |
| | Control of Innoculum | | Log | % |
| Microorganism | Population | Log | Reduction | Reduction |
| Herpes Simplex | 1.13 × 10⁶ | 6.05 Log₁₀ | >5.35 Log₁₀ | 99.999% |
| Human | 1.13 × 10⁶ | 6.05 Log₁₀ | >5.35 Log₁₀ | 99.999% |
| Papillomavirus | | | | |
| Bovine | 1.13 × 10⁶ | 6.05 Log₁₀ | >5.35 Log₁₀ | 99.999% |
| Diarrhea virus | | | | |
| E. coli | 3.7 × 10⁶ | 6.57 Log₁₀ | >5.87 Log₁₀ | 99.999% |
| K. pneumoniae | 1.46 × 10⁶ | 6.16 Log₁₀ | >5.46 Log₁₀ | 99.999% |
| MRSA | 3.79 × 10⁶ | 6.59 Log₁₀ | >5.89 Log₁₀ | 99.999% |
| VRE | 1.62 × 10⁶ | 6.21 Log₁₀ | >5.51 Log₁₀ | 99.999% |
| S. aureus | 1.13 × 10⁶ | 6.05 Log₁₀ | >5.35 Log₁₀ | 99.999% |
| E. coli NDM-1 | 1.47 × 10⁶ | 6.17 Log₁₀ | >5.47 Log₁₀ | 99.999% |
| S. aureus | 3.5 × 10⁶ | 6.54 Log₁₀ | >5.84 Log₁₀ | 99.999% |
| Linezolid | | | | |
| Resistant | | | | |
| Trichophyton | 4.2 × 10⁶ | 5.62 Log₁₀ | >4.92 Log₁₀ | >99.99% |
| mentagrophytes | | | | |
| S. pyogenes | 4.7 × 10⁵ | 5.67 Log₁₀ | >4.97Log₁₀ | >99.99% |
| E. coli | 1.86 × 10⁶ | 6.27 Log₁₀ | >5.57 Log₁₀ | 99.999% |
| Carbapenem | | | | |
| Resistant | | | | |

A 0.05% HOCl aqueous solution is intended for the cleansing and removal of the most common bacteria and viruses associated with skin-to-skin sexually transmitted diseases (pre and post sexual activity) to promote general sexual hygiene. The 0.05% HOCl aqueous solution meets all requirements of USP <51> Antimicrobial Effectiveness Testing.

Table 3. below represents the efficacy of 0.046% aqueous hypochlorous acid solution in the presence of high organic load. ASTM E2315 Time Kill Assay for Antimicrobial Agents. The study was performed in ATS labs in Minnesota recommended by the U.S. FDA's Tentative Final Monograph for OTC Healthcare Antiseptic Drug. Table 3. shows an efficacy of 99.999% for all microorganisms tested at a concentration of 0.046% in 30 seconds. Additional times were evaluated: 1 minute, 5 minutes, and 10 minutes, all with and without organic soil (Using 10% whole milk and 5% Fetal Bovine Serum). The trend in the efficiency is maintained under the same proportions.

TABLE 3

| | Efficacy at 30 seconds | | | |
|---|---|---|---|---|
| | | | After treatment | |
| | | | with 0.046% HOCl | |
| Antiseptic Ability | | | aqueous solution | |
| HOCl solution | Control of Innoculum | | Log | % |
| Microorganism | Population | Log | Reduction | Reduction |
| A. baumanii | 7.1 × 10⁶ | 6.85 Log₁₀ | >6.15 Log₁₀ | 99.999% |
| E. faecium | 1.05 × 10⁶ | 6.02 Log₁₀ | >5.32 Log₁₀ | 99.999% |
| K. pneumoniae | 1.94 × 10⁶ | 6.29 Log₁₀ | >5.59 Log₁₀ | 99.999% |
| NDM-1 | | | | |
| Candida | 7.7 × 10⁶ | 5.89 Log₁₀ | >5.19 Log₁₀ | 99.999% |
| albicans | | | | |
| Enterobacter | 4.0 × 10⁶ | 6.60 Log₁₀ | >5.90 Log₁₀ | 99.999% |
| aerogenes | | | | |
| Haemophilus | 3.2 × 10⁵ | 5.51 Log₁₀ | >4.81 Log₁₀ | >99.99% |
| influenzae | | | | |
| K. oxytoca | 1.99 × 10⁶ | 6.30 Log₁₀ | >5.50 Log₁₀ | 99.999% |
| K. pneumoniae - | 2.90 × 10⁶ | 6.46 Log₁₀ | >5.76 Log₁₀ | 99.999% |
| ESBL | | | | |
| P. mirabilis | 2.26 × 10⁶ | 6.35 Log₁₀ | >5.65 Log₁₀ | 99.999% |
| P. aeruginosa | 4.6 × 10⁶ | 6.66 Log₁₀ | >5.96 Log₁₀ | 99.999% |
| S. marcenscens | 1.8 × 10⁶ | 6.21 Log₁₀ | >5.37 Log₁₀ | 99.999% |
| S. Epidermidis | 1.98 × 10⁶ | 6.30 Log₁₀ | >5.60 Log₁₀ | 99.999% |

### Example 2. Test of Viral Inactivation (Active Virucide)

Studies on the virucidal ability of a 0.04% HOCl aqueous solution was tested according to the “Standard Test Method for Efficacy of Antimicrobial Agents Against Viruses in Suspension” (ASTM, American Standard for Testing and Materials, E1052-96 Reapproved in 2002). The study was conducted at the University of Bosque where the virucidal efficacy of the test solution was tested against the three viruses: Herpes Simplex Virus type 2 (HSV-2, a DNA virus), Vesicular Stomatitis Virus (VSV, a RNA Virus), and Enterovirus-71 (EV-71, a RNA Virus). The study results are in Table 4 and it was concluded that the test solution eliminated each virus with high efficacy while demonstrating no adverse effects to the host cells.

TABLE 4

| | Efficacy at 30 seconds | | |
|---|---|---|---|
| | | After treatment | |
| | | with 0.04% HOCl | |
| | | aqueous solution | |
| Antiseptic Ability | Control of Innoculum | Log | % |
| Microorganism | Population | Reduction | Reduction |
| Herpes Simplex | 3.4 × 10⁷ | 7.53 | 99.9999% |
| Virus (HSV type 2) | | | |
| Vesicular Stomatitis | 4.6 × 10⁷ | 7.66 | 99.9999% |
| Virus (VSV) | | | |
| Enterovirus-71 (EV-71) | 6.91 × 10⁴ | 4.83 | 99.998% |

### Example 3. Safety Data

Studies on low concentrations of hypochlorous acid have provided no evidence of absorption of the compound through intact skin. Thus, the potential for producing skin reactions is extremely low.

Table 5. contains the results of several acute and sub-chronic toxicity tests.

TABLE 5

| | | Exposure/ | |
|---|---|---|---|
| Test | Animal Model | Dose | Results |
| Acute Oral | Wistar Rats | 5000 mg/Kg of | The acute median lethal dose LD₅₀ |
| Toxicity | | HOCl Solution | of 0.05% concentration HOCl |
| | | | aqueous solutions was |
| | | | demonstrated to be greater than |
| | | | 5000 mg/kg of body weight of |
| | | | HOCl solution for both males and |
| | | | females. |
| Acute Dermal | Albino New | 5000 mg/Kg of | The Acute Lethal Dose was |
| Toxicity | Zealand Rabbits | HOCl Solution | demonstrated to be greater than |
| | | | 5000 mg/kg body weight for males |
| | | | and females. During postmortem |
| | | | macroscopic evaluations, no |
| | | | abnormalities were revealed in any |
| | | | test animal. |
| Acute | Young Adult | Gravimetric | Animals All animals survived |
| Inhalation | Wistar Rats | Chamber | exposure to the test atmosphere. |
| Toxicity | | concentration | The Acute Inhalation LC₅₀ is |
| | | 2.07 mg/L of | greater than 2.07 mg/L of HOCl in |
| | | HOCl | male and female rats. |
| Acute Eye | Albino New | 0.1 mL of a | Observations for ocular irritations |
| Irritation | Zealand Rabbits | 5000 ppm | were made at 1, 2, 3, 4, 7, 14, 21 |
| Study | | solution of | days. was determined not to be an |
| | | hypochlorous | irritant |
| | | acid was used. | |
| | | The eyes of the | |
| | | animals were | |
| | | not washed after | |
| | | the administration | |
| | | of the test | |
| | | substance | |
| Sub. Chronic | Young adult | 1000 mg/Kg | No mortalities, considered |
| Toxicity. 90 | Wistar Rats | Body weight | non-toxic |
| days. | | | |
| Teratogenicity | Females and | Oral | No mortalities, considered |
| | Pregnant Wistar | administration | non-toxic |
| | rats | of | |
| | | 1000 mg/Kg/day | |

Table 5 is showing the safety profile of 0.05% HOCl concentration aqueous solutions. These studies meets the requirements of 40 CRF part 160: U.S. EPA (FIFRA), 1989; OECD, Principles of GLP (as revised in 1997) published in ENV/MC/CHEM (98)17, OECD, paris 1998 and all GLP requirements of the 40 CFR 792: U.S. EPA standards.)

The extensive biocompatibility and animal testing that have been performed supports the safety and efficacy of the 0.05% hypochlorous acid solution. All of the testing showed that the product functions as intended without adverse effects to the skin or mucous membranes. Thus the results show the 0.05% hypochlorous acid solution is safe for the indicated use for the cleansing and removal of most common bacteria and viruses associated with skin-to-skin sexually transmitted diseases (pre- and post-sexual activity) to promote general sexual hygiene. Due to the safety of the solution, the hypochlorous acid solution can be used repeatedly without altering or changing the properties of the skin and mucous membranes.

### Example 4. Use as a Sexual Skin Cleanser Aqueous Solution

A 0.05% HOCl concentration aqueous solution is provided in a high density polyethylene bottle of 10 milliliters with a fine mist sprayer to distribute a light and targeted mist for even application.

GENERAL DIRECTIONS FOR USE: Expense about 4-12 sprays, more specifically about 4-6 sprays of the 0.05% HOCl aqueous solution on the desired area and wait up to 30 seconds to disinfect the area and promote general cleanliness to prevent common infections related to sexual intercourse or sexual contact.

Skin Cleansing: Before sexual contact/activity expense 4-6 sprays, specifically about 5 sprays in the topical (external) genital area with the 0.05% HOCl aqueous solution directly. Let air dry (30 seconds)—no rinsing is necessary. Repeat the application as necessary during sexual intercourse. After sexual activity, expense the 0.05% HOCl aqueous solution as necessary, e.g. 4-6 sprays, in the topical (external) genial area directly to cover genital topical environment. Wait at least 30 seconds. Remove the solution and sexual fluids with a paper towel. Apply 5 extra sprays of the 0.05% HOCl aqueous solution after removing foreign material present in the genital area. Let air dry for 30 seconds—no rinsing is necessary.

Repeat application as described above before each further session of sexual contact.

### Example 5. Biocompatibility Study

The 0.05% HOCl concentration aqueous solution is biocompatible and found to have a high safety profile. Over 20,000 patients have been treated with the solution with zero reported cases of hypersensitivity. The solution is pH controlled for biocompatibility, formulated for use on sensitive mucous membrane areas of the body, is non-antibiotic, non-irritating, non-allergenic, non-invasive, and non-toxic. No adverse effects against cellular growth in the patient.

### Example 6. Clinical Study—Biocompatibility

The biocompatibility of the 0.046% HOCl concentration aqueous solution was tested in a three way controlled and randomized single blind clinical trial. Patients were randomized into three groups (60 patients per group). The antiseptic efficacy and biocompatibility of the test solution vs. povidone-iodine vs. chlorhexidine (Chlorhexidine Gluconate—CHG) in surgical—clean and clean contaminated—open wounds was evaluated in 180 patients admitted to the surgical facilities of Erasmo Meoz Hospital in Cucuta, Colombia.

Study results show that there were no infected patients in the groups treated with the test HOCl solution. There were two infected patients in the chlorhexidine group after five days follow-up, and five infected patients in the iodine group after three days of use.

Given that the wounds were classified as clean and clean contaminated, the appearance of infection in the groups of CHG and Iodine may be because these solutions are not well-tolerated by the body for prolonged use. Additionally, the wounds treated with iodine and CHG showed dried edges and no promising color in wound bed, indicating irritation and toxicity. In contrast, the wounds treated with the test HOCl aqueous solution showed define edges, good color and hydrated wound beds, resulting in effective biocompatibility.

### Example 7. Clinical Studies

### Treatment of Skin Ulcers

A skin ulcer is a type of wound developed on the skin caused by a health problem such as an infection, by a pressure sore, or by blood circulation problems. Herpes simplex virus and syphilis are the most common causes of genital ulcers in the United States. Other infectious causes include chancroid, lymphogranuloma venereum, granuloma inguinale (donovanosis), secondary bacterial infections, and fungi. Venous skin ulcers are caused by poor blood circulation from the legs, such as from venous insufficiency. Arterial skin ulcers happen when artery disease is present.

The global incidence of genital ulcer disease is estimated to be more than 20 million cases annually. HSV types 1 and 2 are the most common causes of genital ulcers in the United States, followed by syphilis and chancroid (Am. Fam. Physician. 1; 85(3):254-262, Diagnosis and Management of Genital Ulcers). The time required for complete healing depends on the size and type of the ulcer; large ulcers may take over a month to heal. One in five women and one in six men 14 to 49 years of age have genital HSV type 2 infection.

A non-randomized, non-placebo controlled study involving 822 patients with a variety of ulcers was conducted. These patients were treated with HOCl aqueous solution (0.046%) as the only therapeutic intervention and followed the patients for six months to evaluate ulcer closure as a marker of therapeutic success.

HOCl aqueous solution showed benefits as an antimicrobial and in facilitating tissue regeneration, with a closing index of 97% for venous ulcers at 6 months and up 75% in the closure of mixed ulcers, even when a surgical procedure was required. HOCl aqueous solution is an effective and easy to handle locally administered product in relation to OTC/at home care. The test solution
Decreased the local infection rate for all ulcers.
Decreased ulceration time and recurrence rate down to 2.7% a year.
Is an excellent adjuvant to the regular therapy.
Easy to use and it keeps the ulcer disinfected and hydrated.
No interference with antibiotics administered orally.

### Prevention of Sexual Infections

A randomized double blind, placebo controlled clinical trial was conducted evaluating the efficacy of a HOCl (0.046%) aqueous solution application as a sexual cleanser. The purpose of this study was to evaluate the real world use and effect of the solution in a group of 47 willing participants located in Cartagena, Colombia. These willing participants were all active sex workers, whom on a weekly basis, were actively engaged in sexual intercourse with the general public. This demographic was selected as a “high risk” group of individuals that frequently encounter many forms of sexually transmitted infections. Throughout the study, the “HOCl test” group was compared with the “placebo” group in an effort to gauge the efficacy of prevention and treatment for contact-to-contact sexually transmitted infections as well as common infections related to sex. There were 24 patients in the HOCl test group and 23 patients in the placebo group. The patients were required to complete extensive evaluations regarding their sexual health and lifestyles. The duration of the study was six months.

The following results carry a 85% confidence level as they are dependent on a final round of patient testing and examination. The HOCl test group had zero cases of reported contact-to-contact STIs where placebo group had three reported cases, a 13% increase. The HOCl test group reported one case (4.2% increase) of a urinary tract infection compared to five cases reported in the placebo group (21.7% increase). The HOCl test group reported zero cases of yeast infections compared to the placebo group that reported two cases, an 8.7% increase. The HOCl test group reported two cases of rashes from irritation (8.3% increase) compared to the placebo group that reported six cases (26.1% increase). The HOCl test group reported a total of three infections (13.5% increase) compared to the placebo group that reported a total of sixteen cases (69.6% increase). 93% of users reported that having an additional safe sex product made them feel more confident about sexual health while 82% reported that it would decrease their anxiety of STIs and sexual health in general. No significant change of the pH of the vagina was reported in all 47 patients.

When compared to the placebo group, the HOCl test group demonstrated viable efficacy for prevention of contact-to-contact STIs. Additionally, the HOCl test group showed robust efficacy in preventing and treating common infections related to sex, including urinary tract infections, yeast infections, and general skin rashes or irritation. This study concludes base line efficacy for the aqueous HOCl solution.

### Fournier's Gangrene

Fournier's gangrene is a bacterial infection of the skin that affects the genitals and perineum (i.e., area between the scrotum and anus in men and between the vulva and anus in women). The disease develops after a wound or abrasion becomes infected. A combination of anaerobic microorganisms (e.g., staphylococcal) and fungi (e.g., yeast) causes an infection that spreads quickly and causes destruction (necrosis) of skin, tissue under the skin (subcutaneous tissue), and muscle. Staphylococcal bacteria clot the blood, depriving surrounding tissue of oxygen. The anaerobic bacteria thrive in this oxygen-depleted environment and produce molecules that instigate chemical reactions (enzymes) that further the spread of the infection. Fournier's gangrene can be fatal if the infection enters the bloodstream.

A HOCl (0.046%) aqueous solution was preformed in a four step surgical process to a patient that was infected with Fournier's gangrene. Skin was removed from the infected area before administering the HOCl solution on the infected areas in a 24 hours period. Following application, skin graphs were placed over the surgical wound for rehabilitation process. The result ended in prevention of further spread of the disease as well as prevention of amputation and additional complications.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for promoting sexual hygiene in a subject, comprising:
topically applying an effective amount of a hypochlorous acid composition to the genitals and surrounding skin of a subject exposed to sexual contact, wherein the topically applying occurs before and immediately after sexual activity; and
allowing the hypochlorous acid composition to remain on the skin.

2. A method for eliminating contact-to-contact STDs in a subject, comprising:
topically applying an effective amount of a hypochlorous acid composition to the genitals and surrounding skin of a subject exposed to sexual contact/fluids, wherein the topically applying occurs before and immediately after sexual activity; and
allowing the hypochlorous acid composition to remain on the skin for a time sufficient to kill contact-to-contact STDs and/or to inhibit absorption of a contact-to-contact STD into the subject via the skin.

3. A method of cleansing pre- and post-sexual activity transmitted microorganisms, pathogens, viruses, bacteria, and/or bacterial spores, comprising:
topically applying an effective amount of a hypochlorous acid composition to the genitals and surrounding skin of a subject exposed to sexual contact, wherein the topically applying occurs before and immediately after sexual activity; and
allowing the hypochlorous acid composition to remain on the skin for a time sufficient for a greater than 99% reduction in microorganisms, pathogens, viruses, bacteria, and/or bacterial spores before and after sexual contact.

4. The method of claim 1, wherein the allowing the hypochlorous acid composition to remain on the skin is for 30 seconds or more.

5. The method of claim 1, wherein the hypochlorous acid composition is a liquid, a gel, a lotion, a cream, or a foam.

6. The method of claim 1, wherein the hypochlorous acid composition is an aqueous solution comprising about 0.005 to about 0.06%, about 0.01 to about 0.05, about 0.02 to about 0.046%, or about 0.03 to about 0.04% hypochlorous acid by volume.

7. The method of claim 1, wherein the hypochlorous acid composition is an aqueous solution with a pH of about 2 to about 6, about 3 to about 5, or about 3.5 to about 4.5.

8. The method of claim 1, wherein the hypochlorous acid composition is an aqueous solution comprising about 0.005 to about 0.06% hypochlorous acid by volume, and the aqueous solution has a pH of about 3.5 to about 4.5.

9. The method of claim 1, wherein the hypochlorous acid composition is an aqueous solution comprising about 0.01 to about 0.05% hypochlorous acid by volume, and the aqueous solution has a pH of about 3.5 to about 4.5.

10. The method of claim 1, wherein the hypochlorous acid composition is an aqueous solution comprising about 0.04 to about 0.05% hypochlorous acid by volume, and the aqueous solution has a pH of about 3.5 to about 4.5.

11. The method of claim 1, wherein the hypochlorous acid composition is an aqueous solution comprising about 0.04 to about 0.046% hypochlorous acid by volume, and the aqueous solution has a pH of about 5.0 to about 6.0.

12. The method of claim 1, wherein the hypochlorous acid composition has an oxidation-reduction potential of about 900 to about 1300 mV, about 1000 to about 1250 mV, or about 1100 to about 1200 mV.

13. The method of claim 1, wherein the hypochlorous acid composition is formulated as a liquid spray or in the form of a pre-moistened towel or wipe saturated with the hypochlorous acid composition.

14. The method of claim 1, wherein the hypochlorous acid composition is applied in an amount sufficient to saturate the genitals and surrounding skin of the subject exposed to sexual contact.

15. The method of claim 1, wherein the hypochlorous acid composition is effective to provide a greater than 99% reduction in microorganisms, pathogens, viruses, bacteria, and/or bacterial spores before and after sexual contact, wherein the microorganisms, pathogens, viruses, bacteria, and/or bacterial spores include Aspergillus niger, Bovine Diarrhea virus, Campylobacter rectus, Candida albicans, Enterobacter cloacae, Enterococcus faecalis, Enterococcus faecium, Escherichia coli, Hepatitis B and C virus, Herpes simplex virus type-2 and type-1, human immunodeficiency virus (HIV), human papilloma virus (HPV), Klebsiella oxytoca, Klebsiella pneumoniae, methicillin-resistant staphylococcus aureus, trichomoniasis, molluscum contagiosum, Pseudomona aeruginosa, Salmonella enteritidis, Staphylococcus aureus, Staphylococcus epidermidis, Streptococcus pyogenes, syphilis, Treponema pallidum, Trichophyton mentagrophytes, vancomycin-resistant enterococci, or a combination thereof.

16. The method of claim 1, wherein the hypochlorous acid composition is effective to provide a greater than 99% reduction in microorganisms, pathogens, viruses, bacteria, and/or bacterial spores before and after sexual contact, wherein the microorganisms, pathogens, viruses, bacteria, and/or bacterial spores include Herpes simplex virus, human papilloma virus (HPV), or a combination thereof.

17. The method of claim 1, wherein the subject has intact skin in the genital area and surrounding skin.
